(19) United States
(12) Patent Application Publication
(10) Pub. No.: US 2004/0138699 A1
(43) Pub. Date: Jul. 15, 2004

(54) Beauty tape - over night non surgical face lift to reduce fine lines on face, and wrinkles

(76) Inventor: Margaret A. Lish, Malta, NY (US)

Correspondence Address: Margaret A. Lish, 1030 Laural Lane, Malta, NY, 12020, US

(21) Appl. No.: 10/340,964

(22) Filed: Jan. 13, 2003

(51) Int. Cl. A61F 5/08

(52) U.S. Cl. 606/204.35

(57) ABSTRACT

Beauty tape:

Will be an opaque or clear tape for the face with seven pieces that are designed for seven areas of the face that are prone to wrinkles. It will be made out of a hypoallergenic and breathable material.

Consisting of piece number 1 will be for the forehead. 2 and 3 will be for the temples. 4 will be for the top lip, 5 for below bottom lip, 6 on right cheek, next to the nose and mouth, and 7 will be placed on the left cheek next to the nose and mouth. It will be a very fine textured tape with small holes.

Beauty Tape will be flesh tone or clear tape, with seven pieces that are designed for seven areas of the face that are prone to wrinkles, it will be made with a hypoallergenic and breathable material.

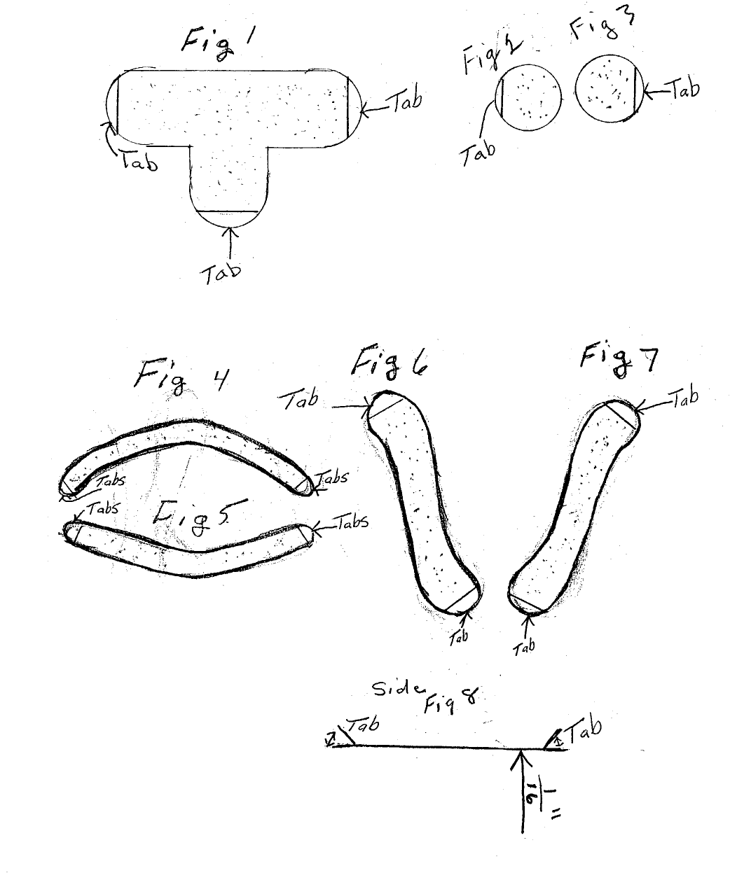

FIG. 1. Will be placed on forehead

FIGS. 2&3. Will be placed on temples

FIG. 4. Will be placed above top lip

FIG. 5. Will be placed below bottom lip

FIG. 6. Will be placed on right cheek next to nose and mouth

FIG. 7. Will be placed on left cheek next to nose and mouth

FIG. 8. Side fig.

1. Beauty Tape:
Will be specially designed for the face it will hold and relax the facial muscles when you sleep to reduce fine lines.
